SEQUENCE LISTING (22 sequences)

SEQ ID NO: 1 - protein, 138 aa, Bradyrhizobium japonicum
QSVNWTWTNQYGSTLAITSFNSNTGAITGTYTNNAANSCDEGKPQGVTGWLAYGNTGTAISFSVNFLGCGSTTVWTGQLNNATGFQGLWYLSLAEAVAWNGISAGADTFTFSSGDKALLTKSGVDLKAGSEKLSNTKK

SEQ ID NO: 2 - protein, 118 aa, Bradyrhizobium japonicum
QSVNWTWTNQYGSTLAITSFNSNTGAITGTYTNNAANSCDEGKPQGVTGWLAYGNTGTAISFSVNFLGCGSTTVWTGQLNNATGFQGLWYLSLAEAVAWNGISAGADTFTFSSGDKAL

SEQ ID NO: 3 - DNA, 492 bp, Bradyrhizobium japonicum
atgcgtcacttcaacggaatgctcttggcgatgattgcgtcgacgtccctgatcggacccctgcccgcatatgcgcagtcggtgaactggacctggaccaaccagtatggctcgaccctggccatcaccagcttcaactcgaacaccggcgcgatcacgggcacctacaccaacaacgccgcaaacagttgcgacgagggcaagccgcaaggtgtgacgggttggctggcctacggcaacaccggcacagcgatcagtttctcggtgaacttcctcggctgcggatcgaccaccgtctggacggggcaattgaacaatgcgacgggattccagggattgtggtatctgtccctcgccgaagccgtggcgtggaacggcatcagcgcgggcgccgacacgttcacgttcagcagcggcgacaaggccctcctgaccaagagcggcgtcgatctgaaggccggcagcgagaagctgtcgaacaccaagaagtag

SEQ ID NO: 4 - DNA, 432 bp, Bradyrhizobium japonicum
atgcgtcacttcaacggaatgctcttggcgatgattgcgtcgacgtccctgatcggacccctgcccgcatatgcgcagtcggtgaactggacctggaccaaccagtatggctcgaccctggccatcaccagcttcaactcgaacaccggcgcgatcacgggcacctacaccaacaacgccgcaaacagttgcgacgagggcaagccgcaaggtgtgacgggttggctggcctacggcaacaccggcacagcgatcagtttctcggtgaacttcctcggctgcggatcgaccaccgtctggacggggcaattgaacaatgcgacgggattccagggattgtggtatctgtccctcgccgaagccgtggcgtggaacggcatcagcgcgggcgccgacacgttcacgttcagcagcggcgacaaggccctctaa

SEQ ID NO: 5 - protein, 163 aa, Bradyrhizobium japonicum
MRHFNGMLLAMIASTSLIGPLPAYAQSVNWTWTNQYGSTLAITSFNSNTGAITGTYTNNAANSCDEGKPQGVTGWLAYGNTGTAISFSVNFLGCGSTTVWTGQLNNATGFQGLWYLSLAEAVAWNGISAGADTFTFSSGDKALLTKSGVDLKAGSEKLSNTKK

SEQ ID NO: 6 - DNA, 420 bp, Gallus gallus
gaattccgcaaggagcacacccggctgtccacctgctgcagagatggtgcacgcaacctccccgctgctgctgctgctgctgctcagcctggctctggtggctcccggcctctctgccagaaagtgctcgctgactgggaaatggaccaacgatctgggctccaacatgaccatcggggctgtgaacagcagaggtgaattcacaggcacctacatcacagccgtaacagccacatcaaatgagatcaaagagtcaccactgcatgggacacaaaacaccatcaacaagaggacccagcccacctttggcttcaccgtcaattggaagttttcagagtccaccactgtcttcacgggccagtgcttcatagacaggaatgggaaggaggtcctgaagaccatgtggctgctgcggtcaag

SEQ ID NO: 7 - DNA, 540 bp, Streptomyces avidinii
ccctccgtccccgccgggcaacaactagggagtatttttcgtgtctcacatgcgcaagatcgtcgttgcagccatcgccgtttccctgaccacggtctcgattacggccagcgcttcggcagacccctccaaggactcgaaggcccaggtctcggccgccgaggccggcatcaccggcacctggtacaaccagctcggctcgaccttcatcgtgaccgcgggcgccgacggcgccctgaccggaacctacgagtcggccgtcggcaacgccgagagccgctacgtcctgaccggtcgttacgacagcgccccggccaccgacggcagcggcaccgccctcggttggacggtggcctggaagaataactaccgcaacgcccactccgcgaccacgtggagcggccagtacgtcggcggcgccgaggcgaggatcaacacccagtggctgctgacctccggcaccaccgaggccaacgcctggaagtccacgctggtcggccacgacaccttcaccaaggtgaagccgtccgccgcctccat

SEQ ID NO: 8 - protein, 128 aa, Streptomyces avidinii
AAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 9 - protein, 128 aa, Gallus Gallus
ARKCSLTGKWTNDLGSNMTIGAVNSRGEFTGTYITAVTATSNEIKESPLHGTENTINKRTQPTFGFTVNWKFSESTTVFTGQCFIDRNGKEVLKTMWLLRSSVNDIGDDWKATRVGINIFTRLRTQKE

SEQ ID NO: 10 - protein, 150 aa, Gallus Gallus
MVHATSPLLLLLLLSLALVAPGLSARKCSLTGKWDNDLGSIMTIGAVNDNGEFNGTYITAVADNPGNITRSPLLGIQHKRACQPTFGFTVHWNFSESTSVFVGQCFVDKSGKEVLKTKWLQRLAVDDISDDWKATRVGNNDFTRQRTVEE

SEQ ID NO: 11 - protein, 150 aa, Gallus Gallus
MVHATSPLLLLLLLSLALVAPSLSARKCSLTGEWDNDLGSIMTIGAVNDNGEFDGTYITAVADNPGNITLSPLLGIQHKRASQPTFGFTVHWNFSESTSVFVGQCFVDRSGKEVLKTKWLQRLAVDDISDDWIATRVGNNDFTRQHTVEE

SEQ ID NO: 12 - protein, 150 aa, Gallus Gallus
MVHTTSPLLLLLLLSLALVAPSLSARKCSLTGKWTNNLGSIMTIRAVNSRGEFAGTYLTAVADNPGNIKLSPLLGIQHKRACQPTFGFTVHWNFSESTSVFVGQCFIDRSGKEVLKTKWLQRLAVDDISDDWKATRVGYNNFTRQRTVEE

SEQ ID NO: 13 - protein, 150 aa, Gallus Gallus
MVHTTSPLLLLLLLSLALVAPSLSARKCSLTGKWTNNLGSIMTIRAVNSRGEFTGTYLTAVADNPGNITLSPLLGIQHKRASQPTFGFTVHWNFSESTTVFTGQCFIDRNGKEVLKTMWLLRSSVNDISYDWKATRVGYNNFTRLCTVEE

SEQ ID NO: 14 - protein, 150 aa, Gallus Gallus
MVHATSPLLLLLLLSLALVAPGLSARKCSLTGEWDNNLGSIMTIGAVNDNGEFNGTYITAVADNPGNIKLSPLLGIQHKRACQPTFGFTVHWNFSESTSVFVGQCFVDRSGKEVLKTKWLQRLAVDDISDDWKATRVGYNNFTRQRTVEE

SEQ ID NO: 15 - protein, 150 aa, Gallus Gallus
MVHATSPLLLLLLLSLALVAPGLSARKCSLTGEWDNNLGSNMTIGAVNDNGEFNGTYITAVADNPGNIKLSPLLGIQHKRACQPTFGFTVHWNFSESTSVFVGQCFIDRSGKEVLKTKWLQRLAVDDISDDWKATRVGYNNFTRQRTVEE

SEQ ID NO: 16 - protein, 152 aa, Gallus Gallus
MVHATSPLLLLLLLSLALVAPGLSARKCSLTGKWTNDLGSNMTIGAVNSRGEFTGTYITAVTATSNEIKESPLHGTQNTINKRTQPTFGFTVNWKFSESTTVFTGQCFIDRNGKEVLKTMWLLRSSVNDIGDDWKATRVGINIFTRLRTQKE

SEQ ID NO: 17 - protein, 183 aa, Streptomyces avidinii
MRKIVVAAIAVSLTTVSITASASADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 18 - protein, 148 aa, Xanthomonas campestris
MMCMSMRQYAACVALLGSCVSLAQAAPTCNNPVGEWKNQLGSTLTITAVHTSGQLLGTYISPSGTTGGVYPLVGWFANPVAGSTALSKLPAITFSVQWGNYGSMTAWTGTCDASGGVPAITTVWHLVRTGSQYSWDHMLTNSDVFVPK

SEQ ID NO: 19 - protein, 168 aa, Burkholderia pseudomallei
MPIQEIRAITRKELKMKKTLVSLAMLGAFAQPAWADTSTAPNCQNPIGSWLNELGSTMTIASISGTGAITGTYVSPSGTTGQTFSLSGWFYAAPPANNGLDQVTLVTFSVNWNNTAARYNSITTWSGLCQITNNVPTITALYYYSNAFAQYSWKHVNVGQDIFHPIAP

SEQ ID NO: 20 - protein, 166 aa, Burkholderia mallei
MRCTIVLGIRAASPIKEALARPAPRPGRLPSIHRSGRRNMQRLEHVLRRVKAGTGAPIDFSGTWKNELGSTMRIEQSGDSVSGTYESAVSENGGATSGQLSGYVDGDLIAFVVHWDQFQAITAWVGQGGPGASSDRINTLWQMTQQVEAGEEWASINAGADIFVKT

SEQ ID NO: 21 - protein, 130 aa, Bradyrhizobium japonicum
MKRLLGLLLLAQSFLLSAEAMAQGLPAPSYWKNERGSELLIWSANSGTIQGTFTNHAQGFACQGIPYPAAGSVSPTGLYFVVTFAQCNSFTRWVGTIKGSQMPTSWTLFYVDNKGKPSRLKGGDIFTRVW

SEQ ID NO: 22 - protein, 179 aa, Rhiz
MIITSLYATFGTIADGRRTSGGKTMIRTNAVAALVFAVATSALAFDASNFKDFSSIASASSSWQNQSGSTMIIQVDSFGNVSGQYVNRAQGTGCQNSPYPLTGRVNGTFIAFSVGWNNSTENCNSATGWTGYAQVNGNNTEIVTSWNLAYEGGSGPAIEQGQDTFQYVPTTENKSLLKD

(19) United States
(12) Patent Application Publication
(10) Pub. No.: US 2010/0022401 A1
(43) Pub. Date: Jan. 28, 2010

(54) Avidin-like proteins from symbiotic bacteria

(75) Inventors: Henri Rainer Nordlund, Lempaala (FI); Vesa Hytonen, Ruutana (FI); Markku Kulomaa, Tampere (FI)

Correspondence Address: YOUNG & THOMPSON, 209 Madison Street, Suite 500, Alexandria, VA, 22314, US

(73) Assignee: LICENTIA OY, HELSINKI (FI)

(21) Appl. No.: 11/666,473

(22) PCT Filed: Oct. 28, 2005
(86) PCT No.: PCT/FI2005/000467

§ 371 Date: Oct. 15, 2009

(30) Foreign Application Priority Data
Oct. 29, 2004 (FI) 20041396

(51) Int. Cl. C40B 30/02; C07K 14/435; C12N 15/12; C12N 15/85; C12N 5/10

(52) U.S. Cl. 506/8; 530/350; 536/23.5; 435/320.1; 435/325

(57) ABSTRACT

An isolated protein which is structurally and functionally similar to avidin but with improved properties, such as better affinity towards biotin conjugate, useful immunological properties or faster biotin dissociation rate, compared to avidin and streptavidin.

### FIELD OF THE INVENTION

The present invention relates to novel avidin-like proteins and a method for producing thereof, genes encoding the proteins, and methods for using the proteins and the genes. Specifically, it relates to a native and a truncated high affinity biotin-binding protein originated from Bradyrhizobium japonicum which proteins resemble (strept)avidin structurally and functionally.

### BACKGROUND OF THE INVENTION

Several avidin proteins have been found in bird, reptile and amphibian species (Hertz and Sebrell, 1942; Jones, 1962; Korpela et al., 1981). Those of the bird avidins that have been characterised are relatively similar, though displaying some differences in stability and immunological cross-reactivity when compared to those of chicken avidin (Hytönen et al., 2003; Korpela et al., 1981). In the chicken the avidin gene forms a gene family together with the avidin-related genes (AVR) (Ahlroth et al., 2000). These AVR proteins have recently been produced as recombinant proteins. Their characterisation and comparison with each other and with avidin revealed some differences in the properties of stability, glycosylation and biotin-binding, although the primary amino acid sequences are rather well conserved (Hytönen et al., 2004b; Laitinen et al., 2002). Several Streptomyces strains were studied four decades ago, and the bacterial analog for avidin, streptavidin, was found in a strain, which was given the name S. avidinii (Chaiet and Wolf, 1964). Inspired by these studies, other Streptomycetes have since been studied. So far two new streptavidins have been found in S. venezuelae and have been named accordingly streptavidin v1 and v2. These new forms were found to be almost identical with streptavidin, displaying only one (v1) or five (v2) amino acid substitutions in the core region, and with no observed significance for either the structure or the function of these proteins (Bayer et al., 1995).

Biotin is an essential cofactor in many vital biochemical reactions (Samols et al., 1988; Wood and Barden, 1977). Therefore it is understandable that (strept)avidin can work as a broad-range antimicrobial agent by forming a biotin free zone or protective barrier around an organism or, for example, an egg possessing it (Green, 1975). The biological role of bradavidin could also be protective as it proved to be a high affinity biotin binding protein. If the B. japonicum-containing root nodules on soybeans are found to express, possibly upon injury or infection, and contain or secrete at least a small amount of bradavidin, among other defence proteins and compounds, the plant could be resistant towards many invaders. These could include harmful soil microbes, insects and also higher animals. Experiments on transgenic corn have shown that the expression of avidin in the plant has an enormous impact on the majority of insect pests, particularly at certain developmental stage of the larvae (Kramer et al., 2000; Morgan et al., 1993).

Medical applications of avidin-biotin technology (Wilchek and Bayer, 1990; Wilchek and Bayer, 1999) include, for example, gene therapy (Lehtolainen et al., 2003; Wojda et al., 1999), imaging (Rosebrough, 1996) and targeted drug delivery (Lehtolainen et al., 2002; Räty et al., 2004). In traditional 1-step radioimmunotherapy (RIT) a therapeutic radioactive material is directly linked to a tumor-specific antibody (Beaumier et al., 1991; Klein et al., 1989; Knox et al., 1992). In order to improve the low target/non-target ratio, which is a drawback with this methodology, several improved protocols for delivering tumor cell-targeted radiation, which usually include more steps, have been developed (Boerman et al., 2003). One of the most promising methods is the 3-step pretargeting radioimmunotherapy (PRIT), which includes the following steps: (i) a biotinylated antibody specific for the target tumor cells, (ii) chicken avidin (fast pharmacokinetic clearance) as a clearing agent to remove endogenous biotin and the excess free circulating biotinylated antibodies from the first step, followed by streptavidin (slow pharmacokinetic clearance) which is mainly responsible for avidinylation of the tumor cells, and (iii) biotinylated radioactive material, which binds tightly to the free binding sites of the tetravalent (strept)avidin molecules immobilised by the biotinylated antibodies (Grana et al., 2002; Paganelli et al., 1999; Paganelli et al., 1991).

In addition to chicken avidin and streptavidin, the existing, rather thoroughly characterised avidin protein pool for medical purposes includes poultry avidins, of which duck, goose and ostrich avidin (Hytönen et al., 2003) in particular have been shown in vitro to be potential alternatives for patients who have strong immunological response toward (strept)avidin owing to usually repeated treatments. Some of the AVR proteins (Laitinen et al., 2002) might also prove useable instead of or before (strept)avidin in sequential PRIT treatments, if they turn out to be immunologically different enough in vivo and show no significant crossreactivity with the antibodies elicited in the possible preceding steps.

Furthermore, differences in pharmacokinetics and other properties owing, for example, to varied glycosylation patterns and protein pI (Rosebrough and Hartley, 1996), can be exploited when selecting avidins for specific applications. However, as all these biotin-binding proteins are xenoproteins, they are likely to be antigenic, and therefore cannot be used effectively on successive occasions with the same patient. Therefore, an immediate need exists for new and dissimilar avidins, such as the characterised bradavidin and the others discussed in this report.

### DESCRIPTION OF THE INVENTION

Bradyrhizobium japonicum is an important nitrogen-fixing symbiotic bacterium, which can form root nodules on soybeans. These bacteria have a gene encoding a putative avidin- and streptavidin-like protein, which bears an amino acid sequence identity of only about 30%, over the core regions, with both of them. The inventors produced this protein in E. coli both as the full length wild-type (SEQ ID NO: 1) and as a C-terminally truncated core (SEQ ID NO:2) forms, and showed that it is indeed a high affinity biotin-binding protein which resembles (strept)avidin structurally and functionally.

Here the expression “resembles structurally and functionally” refers to a protein which can fold to form a 3D-structure spatially like that of (strept)avidin and which can act similarly, for example by containing the crucial amino acid residues for substrate binding. However, other parts of the amino acid sequence, or secondary structure, may be substantially different as well as the immunological properties.

Owing to the considerable dissimilarity in the amino acid sequence, however, the avidin-like protein of the invention is immunologically very different, and polyclonal rabbit and human antibodies toward (strept)avidin do not show significant cross-reactivity with it. Therefore this new avidin, named bradavidin, facilitates medical treatments such as targeted drug delivery, gene therapy and imaging, by offering an alternative tool for use if (strept)avidin cannot be used, due to a deleterious patient immune response for example.

In addition to its medical value, bradavidin can both be used in other applications of avidin-biotin technology as well as a source of new ideas when creating engineered (strept)avidin forms by changing or combining desired parts, interface patterns or specific residues within the avidin protein family. Moreover, the unexpected discovery of bradavidin indicates that the group of new and undiscovered bacterial avidin-like proteins may be both more diverse and more common than hitherto thought.

Accordingly, the first aspect of the present invention is an isolated protein that is structurally and functionally avidin-like with improved properties compared to native avidin or streptavidin and avidin-related proteins, AVRs, and an amino acid sequence having 40% or less, preferably about 30% or less homology, with avidin or streptavidin and highly conserved fingerprint having the sequence:
WXN(E/Q/N/D)XGSX(M/L/F)X(I/V)X_{7,12}GX(F/Y)X_{17,36}(F/Y)XVX(F/W)X_{3,10}(S/A)X(T/S)X(W/F)XGX_{5,14}(M/I/F/L)XXX(W/Y)X_{16,21}(D/N)XF,
   wherein X denotes any amino acid residue, alternatives for a certain position are shown in parentheses, and the subscripted numbers indicate the lower and upper limit, respectively, for the length of the X-stretch in question.

Here the highly “conserved fingerprint” describes a systematic and logical arrangement of conserved amino acids on a sequence. This fingerprint has been assembled through studies on tertiary structures and simulated binding interactions of known AVR proteins and their ligands. This pattern fits onto avidin, streptavidin and bradavidin sequences, even though the homology between these three is not very high. The fundamental factor is the location of the key amino acid residues in 3D-space while other residues connected to the protein backbone facilitate the correct foundation to reach these positions. By posing these key residues into accurate secondary arrangement and by appropriately limiting the distance between the fixed positions, allowing only small fluctuation between these essential avidin characteristics, a search string to select sequences fulfilling the requirements can be designed. By searching through databases, for example DNA libraries, the proteins of interest can be selected using this probe. Most of the prospective search hits have a strong potential to be avidins.

Although avidin and streptavidin are structurally and functionally similar, their pharmacokinetic characteristics differ radically (Rosebrough, 1993; Rosebrough and Hartley, 1996; Schechter et al., 1990). Both glycosylation and high pI are thought to cause the rapid clearance of avidin from the blood. It has been found that glycosylation causes the avidin accumulation in the liver, and the high pI is responsible for the avidin accumulation in the kidneys (Yao et al., 1999). Streptavidin, which has a significantly longer plasma-half life when compared to avidin, is known to accumulate in the kidneys (Schechter et al., 1990), possibly via integrin-mediated cell adhesion dependent on an RGD-like domain (Alon et al., 1993). A streptavidin mutant, in which this RGD-like stretch was modified, showed markedly reduced cell adhesion (Murray et al., 2002). Several attempts have been made to modify (strept)avidin in order to change their in vivo accumulation, clearance and immunological properties. Chinol et al. were able to lower avidin accumulation in the kidneys and liver by attaching polyethylene glycol (PEG) groups to avidin (Chinol et al., 1998). Furthermore, these PEGylated avidins were found to be less immunogenic. Also epitope-modified recombinant streptavidins, carrying point mutations, with markedly improved immunological properties, have been generated (Meyer et al., 2001). In another study, deglycosylated and chemically neutralized avidin was found to be superior when compared to wt avidin in brain delivery (Kang and Pardridge, 1994). The better penetration was supposed to be due to the extended circulation time. Analogously, when galactose moieties were chemically attached to streptavidin, its blood clearance was accelerated (Rosebrough and Hartley, 1996). Yao et al. in turn made other peculiar observations. They demonstrated that avidin itself accumulated efficiently in lectin-expressing tumors, whereas streptavidin and chemically neutralized avidin did not exhibit this kind of behaviour (Yao et al., 1998).

The biotin-binding properties of bradavidin were shown to bear more resemblance to streptavidin than avidin. Bradavidin displayed the fastest dissociation rate, when radio-biotin was used in the analysis. Avidin showed clearly the slowest dissociation, whereas the value for streptavidin fell between these two. Moreover, when the ligand was a fluorescent biotin conjugate, avidin was clearly the fastest in dissociation, and streptavidin and bradavidin were nearly identical showing a very slow and small release in the assay. This is in line with a previous study (Pazy et al., 2002), in which streptavidin was proven to be better biotin conjugate binder than avidin. This property, also characteristic of bradavidin, is interesting and renders it a good tool in applications, since the biotin in use is usually a conjugate and thus good affinity is essential. The structural differences in the loop between β-strands 3 and 4 are thought to explain this divergent binding ability of avidin and streptavidin (Livnah et al., 1993; Pazy et al., 2002; Weber et al., 1989). In bradavidin this loop is extraordinary, since it probably contains a cysteine residue, which forms a disulfide bridge with the cysteine residue on the structurally neighbouring loop between β-strands 5 and 6. This interesting motif, potentially on top the entrance of the binding site, could have some effect on the binding parameters described above. It could also explain the fundamental reasons behind them, such as the divergent association rate, which are intended to be studied in greater detail together with the possible crystal structure of bradavidin.

These characteristics are here referred among “improved properties”. These properties are assessed comparing against those known for thoroughly studied chicken avidin (SEQ ID NO:6) and streptavidin (SEQ ID NO:7). Other examples of improved properties are better affinity towards biotin conjugate, faster biotin dissociation rate, useful immunological properties and beneficial protein/protein-, protein/DNA or protein/ligand interaction or a lack of it, compared to avidin and streptavidin. The table 1 in example 3 is informative presenting measured characteristics for avidin, streptavidin and bradavidin.

The existence of the bradavidin gene in the genome of a root nodule symbiotic bacterium, B. japonicum, may be just the first example of other genes producing functionally similar proteins in other plant-related bacteria. It is possible that further study of the root nodules of species other than the soy-bean will reveal a variety of such proteins. According to the 16S ribosomal RNA gene comparison, the strains producing the different streptavidins (S. avidinii and S. venezuelae) described so far share about 97% sequence identity, indicating close evolutionary relationship. On the contrary, B. japonicum is clearly not a close relative of these bacteria, since its 16S rRNA gene bears only about 74% and 77% sequence identity with those of S. avidinii and S. venezuelae, respectively. A straightforward assumption would be that some of the possible new avidins from symbiotic bacteria closely resemble bradavidin, although completely different forms might also be found.

Another aspect of the invention is a gene encoding an avidin-like protein according to SEQ ID NO:3 or 4. Yet another aspect of the invention is a recombinant vector comprising the any of said genes, a transformant obtained by introducing said recombinant vector to a host organism or a recombinant protein produced by the transformant.

In addition to sequence database queries based on whole sequence similarity, an effective string could be obtained from an extensive multiple sequence alignment of different avidins and related biotin-binding proteins (FIG. 5). By using such an avidin fingerprint string containing only certain functionally and structurally necessary amino acid stretches and patterns new avidins could be found in virtually any life form once the sequence data becomes available. This string could also be utilised when designing probes for cDNA or genomic library screening, emphasising the conserved spots, when the actual sequence is unknown.

Therefore, another aspect of the invention is a method for searching avidin-like proteins from databases comprising use of a search string. An example of such a search string is presented in example 5 and its use is illustrated in FIG. 5.

The comparison of some potential avidin-like sequences, with those of the avidin and avidin-related sequences, shown in the multiple sequence alignment (FIG. 5), revealed intriguing details. The first β-strand conserved, W10 (avidin numbering for amino acids and β-strands) is invariably preceded by G8 or S8, with the exception of bradavidin, which has W in that position. This indicates the possibility of other acceptable substitutions in this position. On the bottom of the biotin-binding site is an important ligand contact residue Y33 (Livnah et al., 1993; Weber et al., 1989), which is conserved, excluding the putative avidin from B. japonicum (brad2), which bears the Y to F substitution. In the previous point-mutagenesis studies of streptavidin (Klumb et al., 1998) and avidin (Marttila et al., 2003) the analogous mutation resulted in a 5- and 13-fold increase, respectively, in the dissociation rate constants of the ligands studied. It is possible, therefore, that this putative brad2-protein may exhibit high biotin affinity despite an aberrant residue in this particular position.

The loops connecting β-strands three and four in avid in and streptavidin (FIG. 1) are different from each other. The role of this loop is to form certain hydrogen bonds and other contacts with biotin. It seems that the putative avidins of different origin may also form similar interactions, although this ability cannot be definitively demonstrated without a three-dimensional structure with the ligand.

Moreover, W70 and W97 form part of the hydrophobic cavity of the ligand-binding site in avidin (Livnah et al., 1993). The importance of the equivalent residues in streptavidin for biotin binding has also been experimentally shown by Chilkoti et al. (Chilkoti et al., 1995), who mutated these residues to ala nine and phenylalanine and observed a significant decrease in affinity in the case of the alanine mutants. However, in the case of the phenylalanine substitutions the decreases in the observed affinities were only mediocre. It is, therefore assumed, that the W97Y difference present in the putative Burk_pseudomallei avidin would not radically diminish the biotin affinity of this protein. In the present study it was found that bradavidin is a high-affinity biotin-binding protein, although it has F at position 70 and avidin has W the same position, which further supports the idea that conservation of the complementarity of the binding site and the ligand structure is essential for high affinity (Livnah et al., 1993; Weber et al., 1989).

The most radical differences in the multiple sequence alignment are found in the loop around W110, which plays a central role in ligand binding (Chilkoti et al., 1995; Laitinen, 1999 #32; Laitinen et al., 1999; Laitinen et al., 2003; Livnah et al., 1993; Weber et al., 1989). It could be speculated that the loop structures containing the proline residues in Rhiz and Brad2 sequences (FIG. 5) might be able to form contacts comparable to those formed by the tryptophan in avidin and the others of the studied sequences.

The invention will be further described with reference to the following figures and non-limiting examples 1-5.

### BRIEF DESCRIPTION OF THE FIGURES

FIG. 1 Multiple sequence alignment of the core forms of strept(avidin), brad(avidin) and chicken (avidin). The arrows indicate the location of the successive 5-strands according to the structure of chicken avidin. The cysteine residues in chicken avidin (C4 and C84), which form an intramonomeric disulfide bridge, are shown as bold letters. Similarly the cysteine residues (C39 and C69) in bradavidin are shown in bold, and these too could form an intramonomeric disulfide bridge, although not spatially equivalent to that of chicken avidin. The conserved amino acids are marked below by ‘*’ and strong amino acid group similarity by ‘:’, whereas weaker group similarity is indicated by ‘.’. Biotin-binding residues of avidin and streptavidin are underlined (Livnah et al., 1993).

FIG. 2 Biotin dissociation analysis. (A) The [3H]biotin dissociation rate constant was measured at different temperatures. The values for streptavidin are from Klumb et al. (Klumb et al., 1998). The scale of the Y axis is logarithmic. (B) Release of fluorescent biotin conjugate from avidins was studied as a function of time in the presence of excess D-biotin at 50° C.

FIG. 3 Non-reducing but denaturing SDS-PAGE analysis. Wild-type bradavidin is indicated by wt and the C-terminally truncated form by core. The unit of molecular mass markers indicated by M is kDa.

FIG. 4 Immunological cross-reactivity assay. Patients A-E have been subjected to PRIT treatment using both avidin and streptavidin, whereas the donors of the negative control sera N1 and N2 have not been exposed to avidin or streptavidin. Polyclonal rabbit antibodies toward streptavidin (SA) and avidin (AVD) were also tested for cross-reactivity.

FIG. 5 Multiple sequence alignment of known and candidate avidin-like proteins. The N- and C-terminal signals and extensions are included in this alignment. The conserved amino acids are marked below by an asterisk ‘*’, and strong amino acid group similarity is indicated by a colon ‘:’, whereas weaker group similarity is indicated by a single dot ‘.’. Biotin-binding residues of avidin and streptavidin are underlined (Livnah et al., 1993). Proper avidin search string for database queries can be obtained by selecting or emphasising most of the positions marked below by ‘*’ and ‘:’. Moreover this knowledge can be used as the basis for cDNA and genomic library probe design. In addition, by limiting the distance appropriately between the fixed positions, allowing only small fluctuation between these essential avidin characteristics, most of the prospective hits will be very potential avidins. The new candidate sequences are shown below bradavidin. They were obtained by TBlastn using the bradavidin sequence as the query. Avidin related proteins (AVR) are included because-they have been characterized previously as high affinity biotin-binding proteins among avidin. An example of such an avidin string is:
WXN(E/Q/N/D)XGSX(M/L/F)X(I/V)X_{7,12}GX(F/Y)X_{17,36}(F/W)XVX(F/W)X_{3,10}(S/A)X(T/S)X(W/F)XGX_{5,14}(M/I/F/L)XXX(W/Y)X_{16,21}(D/N)XF. In this string, X denotes any amino acid residue, alternatives for a certain position are shown in parentheses, and the subscripted numbers indicate the lower and upper limit, respectively, for the length of the Xstretch in question.

### EXAMPLES

### Example 1

### Production and Purification of Bradavidin

The gene coding for bradavidin (DBJ AP005955.1) was amplified by PCR using B. japonicum genomic DNA as a template, and extended using SES-PCR (Majumder, 1992) to include attL recombination sites at both ends (Hartley et al., 2000). Two constructs were generated: the full length wild-type (138 amino acid residues, SEQ. ID NO:1) and a C-terminally truncated core form (118 amino acid residues, SEQ ID NO:2). Both constructs contained also their innate signal peptides (25 amino acid residues), which is represented together with the wild type protein (163 amino acid residues) in SEQ ID NO:5. These constructs were then transferred to pBVboostFG vector (Laitinen O. H. et al., manuscript) using the site-specific recombination-based Gateway method (invitrogen). The resulting expression vectors were confirmed to be as designed by DNA sequencing.

E. coli BL-21 (AI) cells (Invitrogen) were used for protein expression as described previously (Hytönen et al., 2004a). The recombinant proteins were isolated from bacterial cell extracts by one-step affinity chromatography on 2-iminobiotin agarose column (Hytönen et al., 2004a). Eluted proteins were analysed by SDS-PAGE and subsequent Coomassie staining of the gels. The proteins appeared to be pure and virtually homogenous, as only one band per lane of the expected size was observed on gels. Protein concentrations were determined using the calculated extinction coefficient 39 380 M-1 cm-1 for both bradavidins at 280 nm (Gill and von Hippel, 1989).

### Example 2

### Primary Structure Analysis

Pairwise sequence alignments were done using the Needle program from the EMBOSS (European Molecular Biology Open Software Suite) package and the ClustalW program was used to generate the multiple sequence alignment (Thompson et al., 1994). The theoretic biochemical properties were determined using the ProtParam program (Gill and von Hippel, 1989). The putative signal peptide cleavage site was determined by the SignalP 3.0 program (Bendtsen et al., 2004).

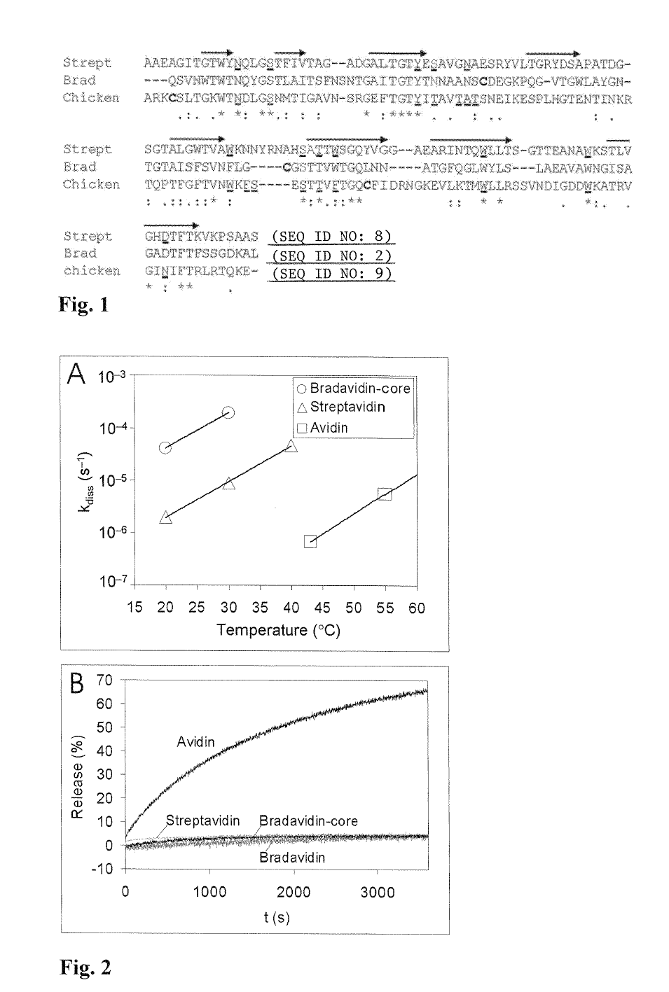

Pairwise sequence alignment for mature core regions of avidin and bradavidin revealed that 29.2% of the amino acids are identical and 39.2% similar, whereas with streptavidin these values are 30.2% and 41.7%, respectively. Interestingly, when avidin and streptavidin are compared equivalently the values obtained, 31.9% and 45.2%, are only slightly higher. Multiple sequence alignment of streptavidin, bradavidin and avidin (FIG. 1) revealed that most of the conserved residues are directly involved in biotin binding or are structurally important characteristics in the avidin protein family (Livnah et al., 1993; Weber et al., 1989). Over the plausible biotin-binding residues bradavidin bears a slightly closer resemblance to streptavidin than avidin. Bradavidin has two cysteine residues which, according to the known avidin structure (Livnah et al., 1993), could form an intramonomeric disulfide bridge spatially different from that in chicken avidin, whereas streptavidin is devoid of cysteines. In line with this, only monomeric forms were observed in the SDS-PAGE samples boiled in sample buffer without the reducing agent β-mercaptoethanol (FIG. 3), indicating that bradavidin does not have intermonomeric disulfide bridges analogous to those present in engineered avidin forms (Nordlund et al., 2003; Reznik et al., 1996).

### Example 3

### Analysis of Function and Properties

Ligand binding properties were studied both by [3H]biotin assay and fluorescent biotin conjugate assay. The dissociation rate constant of [3H]biotin (Amersham) from the bradavidins and avidin was measured at various temperatures as described in detail previously (Klumb et al., 1998). According to the results, (Table I, FIG. 2) the faster dissociation rate measured from bradavidin indicates weaker affinity toward the radiobiotin than those of avidin and streptavidin. The dissociation rate of a fluorescent biotin conjugate (ArcDia BF560™-biotin) was measured as previously described (Hytönen et al., 2004a) at 50° C. However, bradavidin showed a clearly slower rate of fluorescent biotin displacement than that of avidin, thus proving to be almost as extreme biotin conjugate binder as streptavidin (Pazy et al., 2002).

The purified proteins were analysed by gel filtration using a Shimadzu HPLC instrument equipped with a Superdex 200 HR 10/30 column (Amersham Pharmacia Biotech, Uppsala, Sweden) with 50 mM Na-carbonate buffer (pH 11) with 150 mM NaCl as the liquid phase. The column was calibrated using a marker mixture (thyroglobulin, IgG, ovalbumin, myoglobin, vitamin B-12; Bio-Rad Laboratories, Hercules, Calif., U.S.A) and bovine serum albumin (Roche Diagnostics, Mannheim, Germany) as molecular mass standards.

Gel filtration chromatography showed that bradavidin is a homogenous tetramer and both forms appeared as a single symmetrical and sharp peak on the chromatograms. SDS-PAGE stability analysis confirmed the tetrameric appearance. These quaternary structures showed comparable stability with those of avidin and streptavidin (Table I).

The thermal stability characteristics of the proteins were studied by a SDS-PAGE based method as previously described in detail by (Bayer et al., 1996).

The apparent molecular mass is indicated and followed by the theoretical mass in brackets. Transition temperature (Tᵣ) indicates the temperature in which half of the protein is tetrameric and half monomeric in the absence (first value) and presence (second value) of biotin. In addition to the measured dissociation rate constant (k_{diss}), the release percentage of the fluorescent biotin conjugate in one hour in the presence of excess free biotin is indicated. Calculated isoelectrical point (pI) and the number of cysteine residues per monomer are also indicated.

TABLE I
Protein characteristics

| | Gel filtration | Heat treatment | | Fluorescent | Release | | |
|---|---|---|---|---|---|---|---|
| | HPLC | SDS-PAGE | | biotin k_{diss} | 1 h | | Cysteine |
| Protein | KDa | Tᵣ (° C.) | | s⁻¹ | % | PI | residues |
| Bradavidin- | 45.3 (49.2) | 70 | 85 | 1.2 × 10⁻⁵ | 4.4 | 4.1 | 2 |
| core | | | | | | | |
| Bradavidin | 50.0 (57.5) | 65 | 85 | 1.5 × 10⁻⁵ | 4.7 | 6.3 | 2 |
| Streptavidin | 51.1 (53.4) | 72^{a} | 100^{a} | 7.2 × 10⁻⁶ | 5.1 | 6.1 | 0 |
| Avidin | 64.0 (63.1)* | 58^{a} | 100^{a} | 2.7 × 10⁻⁴ | 71.5 | 9.5 | 2 |

*Including the sugar moiety, which comprises about 10% of the mass (Bruch and White, 1982; Green, 1975)
^{a}These values were obtained from Bayer et al. (Bayer et al., 1996)

### Example 4

### Antibody Recognition

Serum samples from cancer patients exposed to avidin and streptavidin were used to compare the immunological properties of the avidins. The serum samples as well as the negative control sera from persons not exposed to (strept)avidin were obtained from the Division of Nuclear Medicine, European Institute of Medicine, Milan, Italy. The analysis was performed similarly as described previously (Hytönen et al., 2003): Immobilizer™ Amino-plates (Nalge Nunc Int.) were coated with the proteins under study (10 μg/ml) in 100 mM Na-phosphate pH 7.5, agitated for one hour at room temperature and blocked with PBS-T (PBS+Tween 20 0.05% v/v). The serum samples were diluted 1:100 in PBS-T and incubated in the wells for one hour at 37° C. After washing three times with PBS-T, polyvalent anti-human immunoglobulin alkaline phosphatase (AP) conjugate (Sigma) was used as a secondary antibody (dilution 1:6000; 1 h, 37° C.), followed by six washes with PBS-T. Finally, p-nitrophenyl phosphate (1 mg/ml, Sigma) was used as a substrate molecule, and a plate reader was used to measure the absorbance at 405 nm.

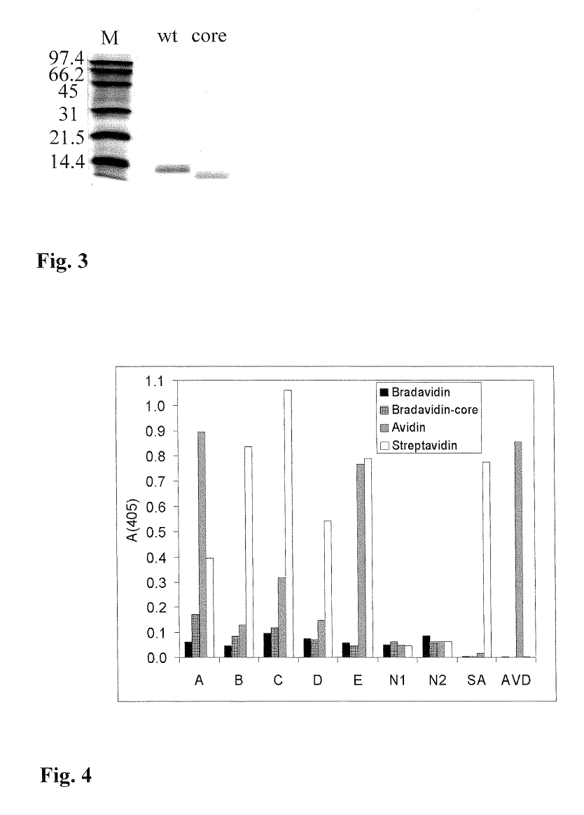

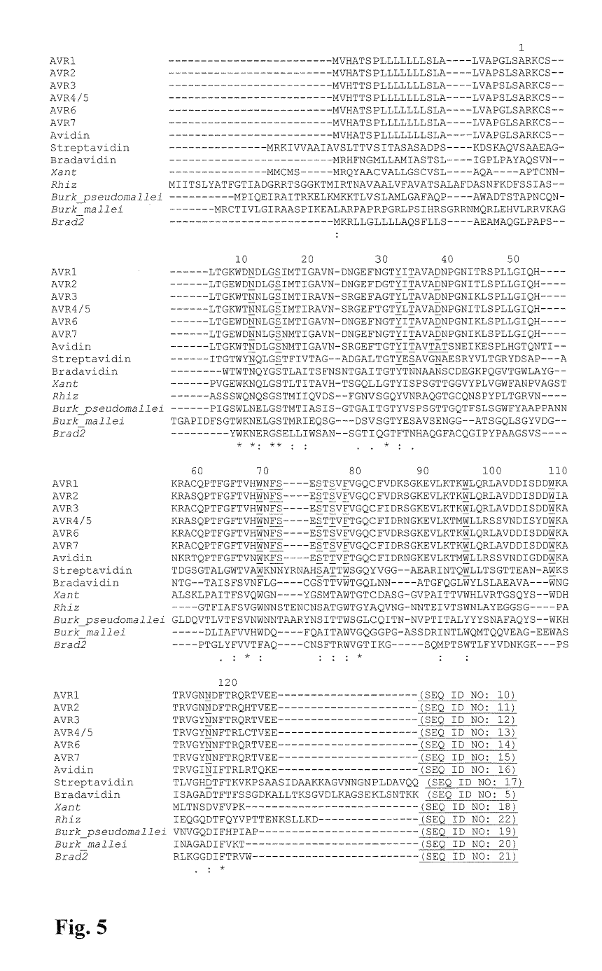

Immunological cross-reactivity of bradavidin with human and rabbit serum antibodies, elicited toward avidin and streptavidin, analysed by an ELISA assay is illustrated in FIG. 4. Samples from cancer patients exposed to avidin and streptavidin recognised avidin and, even more clearly, streptavidin. This may stem not only from the number and extent of medical treatments but also from the fact that streptavidin is more antigenic than avidin (Chinol et al., 1998; Paganelli et al., 1997). None of the patient sera showed a significant response toward bradavidin, which clearly indicated that this protein is largely devoid of common epitopes with (strept)avidin. In addition to human samples, polyclonal rabbit antibodies recognised only the protein toward which they had been elicited in the first place.

Proteins were further compared using polyclonal rabbit antibodies produced against avidin (University of Oulu, Finland) and streptavidin (Weissman Institute, Jerusalem, Israel). Proteins were first attached to Immobilizer™ Amino plates as described above and blocked with PBS-T. Antibodies were diluted 1:2000 to PBS-T and applied to the protein-coated plates (1 h, 37° C.). After washing with PBS-T, goat anti-rabbit IgG AP (Bio-Rad Laboratories) diluted 1:2000 in PBS-T was used as a secondary antibody (1 h, 37° C.), and the signal was measured as above.

When bradavidin was probed by polyclonal anti-(strept)avidin rabbit antibodies on western blots, only the positive (strept)avidin controls were detected after immunostaining. Preceding that, when the nitro-cellulose filter was stained with Ponceau S-dye, wild-type bradavidin was clearly visible at the expected location whereas the bradavidin core appeared to be virtually absent from the blot at this stage (data not shown). This behaviour may result from the rather low pI of the core form (Table I), as also suspected, for example, in the case of the acidic natural rubber latex allergen Hev b5 (Akasawa et al., 1996).

### Example 5

### Comparison of New Potential Avidins

A multiple sequence alignment of many known avidin-like proteins (Chaiet and Wolf, 1964; Green, 1975; Laitinen et al., 2002) and some new candidates suggested biotin binding capability for the open reading frames from Xanthomonas campestris (GenBank AE012315.1), Rhizobium etli (GenBank U80928.4), Bradyrhizobium japonicum (another candidate in addition to bradavidin, DBJ AP005940.1), Burkholderia pseudomallei (EMB BX571965.1) and Burkholderia mallei (GenBank CP000010.1). The majority of the putative biotin-binding residues, according to the avidin (Livnah et al., 1993) and streptavidin (Weber et al., 1989) structures, were conserved albeit the overall sequence similarity was rather low (FIG. 5). Conserved structural characteristics of the avidin fold (Flower, 1993) were also observed in the sequences, which further supports the assumptions made.

### REFERENCES

Ahlroth, M. K., Kola, E. H., Ewald, D., Masabanda, J., Sazanov, A., Fries, R. and Kulomaa, M. S. (2000) Characterization and chromosomal localization of the chicken avidin gene family. Anim Genet, 31, 367-375.
Akasawa, A., Hsieh, L. S., Martin, B. M., Liu, T. and Lin, Y. (1996) A novel acidic allergen, Hev b 5, in latex. Purification, cloning and characterization. J Biol Chem, 271, 25389-25393.
Alon, R., Bayer, E. A. and Wilchek, M. (1993) Cell adhesion to streptavidin via RGD-dependent integrins. Eur J Cell Biol, 60, 1-11.
Bayer, E. A., Ehrlich-Rogozinski, S, and Wilchek, M. (1996) Sodium dodecyl sulfate-polyacrylamide gel electrophoretic method for assessing the quaternary state and comparative thermostability of avidin and streptavidin. Electrophoresis, 17, 1319-1324.
Bayer, E. A., Kulik, T., Adar, R. and Wilchek, M. (1995) Close similarity among streptavidin-like, biotin-binding proteins from Streptomyces. Biochim Biophys Acta, 1263, 60-66.
Beaumier, P. L., Venkatesan, P., Vanderheyden, J. L., Burgua, W. D., Kunz, L. L., Fritzberg, A. R., Abrams, P. G. and Morgan, A. C., Jr. (1991) 186Re radioimmunotherapy of small cell lung carcinoma xenografts in nude mice. Cancer Res, 51, 676-681.
Bendtsen, J. D., Nielsen, H., von Heijne, G. and Brunak, S. (2004) Improved prediction of signal peptides: SignalP 3.0. J Mol Biol, 340, 783-795.
Boerman, O. C., van Schaijk, F. G., Oyen, W. J. and Corstens, F. H. (2003) Pretargeted radioimmunotherapy of cancer: progress step by step. J Nucl Med, 44, 400-411.
Bruch, R. B. and White, H. B. (1982) Compositional and structural heterogeneity of avidin glycopeptides. Biochemistry, 21, 5334-5341.
Chaiet, L. and Wolf, F. J. (1964) The Properties of Streptavidin, a Biotin-Binding Protein Produced by Streptomycetes. Arch Biochem Biophys, 106, 1-5.
Chilkoti, A., Tan, P. H. and Stayton, P. S. (1995) Site-directed mutagenesis studies of the high-affinity streptavidin-biotin complex: contributions of tryptophan residues 79, 108, and 120. Proc Natl Acad Sci USA, 92, 1754-1758.
Chinol, M., Casalini, P., Maggiolo, M., Canevari, S., Omodeo, E. S., Caliceti, P., Veronese, F. M., Cremonesi, M., Chiolerio, F., Nardone, E., Siccardi, A. G. and Paganelli, G. (1998) Biochemical modifications of avidin improve pharmacokinetics and biodistribution, and reduce immunogenicity. Br J Cancer, 78, 189-197.
Flower, D. R. (1993) Structural relationship of streptavidin to the calycin protein superfamily. FEBS Lett, 333, 99-102.
Gill, S. C. and von Hippel, P. H. (1989) Calculation of protein extinction coefficients from amino acid sequence data. Anal Biochem, 182, 319-326.
Grana, C., Chinol, M., Robertson, C., Mazzetta, C., Bartolomei, M., De Cicco, C., Fiorenza, M., Gatti, M., Caliceti, P. and Paganelli, G. (2002) Pretargeted adjuvant radioimmunotherapy with yttrium-90-biotin in malignant glioma patients: a pilot study. Br J Cancer, 86, 207-212.
Green, N. M. (1975) Avidin. Adv Prot Chem, 29, 85-133.
Hartley, J. L., Temple, G. F. and Brasch, M. A. (2000) DNA cloning using in vitro site-specific recombination. Genome Res, 10, 1788-1795.
Hertz, R. and Sebrell, W. H. (1942) Occurrence of avidin in the oviduct and secretions of the genital tract of several species. Science, 96, 257.
Hytönen, V. P., Laitinen, O. H., Airenne, T. T., Kidron, H., Meltola, N. J., Porkka, E., Hörhä, J., Paldanius, T., Määttä, J. A., Nordlund, H. R., Johnson, M. S., Salminen, T. A., Airenne, K. J., Ylä-Herttuala, S, and Kulomaa, M. S. (2004a) Efficient production of active chicken avidin using a bacterial signal peptide in Escherichia coli. Biochem J, Pt.
Hytönen, V. P., Laitinen, O. H., Grapputo, A., Kettunen, A., Savolainen, J., Kalkkinen, N., Marttila, A. T., Nordlund, H. R., Nyholm, T. K., Paganelli, G. and Kulomaa, M. S. (2003) Characterization of poultry egg-white avidins and their potential as a tool in pretargeting cancer treatment. Biochem J, 372, 219-225.
Hytönen, V. P., Nyholm, T. K., Pentikainen, O. T., Vaarno, J., Porkka, E. J., Nordlund, H. R., Johnson, M. S., Slotte, J. P., Laitinen, O. H. and Kulomaa, M. S. (2004b) Chicken Avidin-related Protein 4/5 Shows Superior Thermal Stability when Compared with Avidin while Retaining High Affinity to Biotin. J Biol Chem, 279, 9337-9343.
Jones, P. D., Briggs, M. H. (1962) Distribution of avidin. Life Sciences, 11, 621-623.
Kang, Y.-S. and Pardridge, W. M. (1994) Use of neutral avidin improves pharmacokinetics and brain delivery of biotin bound to an avidin-monoclonal antibody conjugate. The Journal of Pharmacology and Experimental Therapeutics, 269, 344-350.
Klein, J. L., Nguyen, T. H., Laroque, P., Kopher, K. A., Williams, J. R., Wessels, B. W., Dillehay, L. E., Frincke, J., Order, S. E. and Leichner, P. K. (1989) Yttrium-90 and iodine-131 radioimmunoglobulin therapy of an experimental human hepatoma. Cancer Res, 49, 6383-6389.
Klumb, L. A., Chu, V. and Stayton, P. S. (1998) Energetic roles of hydrogen bonds at the ureido oxygen binding pocket in the streptavidin-biotin complex. Biochemistry, 37, 7657-7663.
Knox, S. J., Goris, M. L. and Wessels, B. W. (1992) Overview of animal studies comparing radioimmunotherapy with dose equivalent external beam irradiation. Radiother Oncol, 23, 111-117.
Korpela, J. K., Kulomaa, M. S., Elo, H. A. and Tuohimaa, P. J. (1981) Biotin-binding proteins in eggs of oviparous vertebrates. Experientia, 37, 1065-1066.
Kramer, K. J., Morgan, T. D., Throne, J. E., Dowell, F. E., Bailey, M. and Howard, J. A. (2000) Transgenic avidin maize is resistant to storage insect pests. Nat Biotechnol, 18, 670-674.
Laitinen, O. H., Airenne, K. J., Marttila, A. T., Kulik, T., Porkka, E., Bayer, E. A., Wilchek, M. and Kulomaa, M. S. (1999) Mutation of a critical tryptophan to lysine in avidin or streptavidin may explain why sea urchin fibropellin adopts an avidin-like domain. FEBS Lett, 461, 52-58.
Laitinen, O. H., Hytönen, V. P., Ahlroth, M. K., Pentikainen, O. T., Gallagher, C., Nordlund, H. R., Ovod, V., Marttila, A. T., Porkka, E., Heino, S., Johnson, M. S., Airenne, K. J. and Kulomaa, M. S. (2002) Chicken avidin-related proteins show altered biotin-binding and physico-chemical properties as compared with avidin. Biochem J, 363, 609-617.
Laitinen, O. H., Nordlund, H. R., Hytönen, V. P., Uotila, S. T., Marttila, A. T., Savolainen, J., Airenne, K. J., Livnah, O., Bayer, E. A., Wilchek, M. and Kulomaa, M. S. (2003) Rational design of an active avidin monomer. J Biol Chem, 278, 4010-4014.
Lehtolainen, P., Taskinen, A., Laukkanen, J., Airenne, K. J., Heino, S., Lappalainen, M., Ojala, K., Marjomäki, V., Martin, J. F., Kulomaa, M. S, and Ylä-Herttuala, S. (2002) Cloning and characterization of Scavidin, a fusion protein for the targeted delivery of biotinylated molecules. J Biol Chem, 277, 85458550.
Lehtolainen, P., Wirth, T., Taskinen, A. K., Lehenkari, P., Leppänen, O., Lappalainen, M., Pulkkanen, K., Marttila, A., Marjomäki, V., Airenne, K. J., Horton, M., Kulomaa, M. S, and Ylä-Herttuala, S. (2003) Targeting of biotinylated compounds to its target tissue using a low-density lipoprotein receptor-avidin fusion protein. Gene Ther, 10, 2090-2097.
Livnah, O., Bayer, E. A., Wilchek, M. and Sussman, J. L. (1993) Three-dimensional structures of avidin and the avidin-biotin complex. Proc Nat) Acad Sci USA, 90, 5076-5080.
Majumder, K. (1992) Ligation-free gene synthesis by PCR: synthesis and mutagenesis at multiple loci of a chimeric gene encoding OmpA signal peptide and hirudin. Gene, 110, 89-94.
Marttila, A. T., Hytonen, V. P., Laitinen, O. H., Bayer, E. A., Wilchek, M. and Kulomaa, M. S. (2003) Mutation of the important Tyr-33 residue of chicken avidin: functional and structural consequences. Biochem J, 369, 249-254.
Meyer, D. L., Schultz, J., Lin, Y., Henry, A., Sanderson, J., Jackson, J. M., Goshorn, S., Rees, A. R. and Graves, S. S. (2001) Reduced antibody response to streptavidin through site-directed mutagenesis. Protein Sci, 10, 491-503.
Morgan, T. D., Oppert, B., Czapla, T. H. and Kramer, K. J. (1993) Avidin and streptavidin as insecticidal and growth inhibiting dietary proteins. Entomol. Exp. Appl., 69, 97-108.
Morpurgo, M., Radu, A., Bayer, E. A. and Wilchek, M. (2004) DNA condensation by high-affinity interaction with avidin. J Mol Recognit, 17, 558-566.
Murray, S., Maraveyas, A., Dougan, T. and Chu, A. C. (2002) Reduction of streptavidin RYDS-mediated renal adhesion by site-directed mutagenesis. Biochim Biophys Acta, 1570, 81-88.
Nordlund, H. R., Laitinen, O. H., Uotila, S. T., Nyholm, T., Hytönen, V. P., Slotte, J. P. and Kulomaa, M. S. (2003) Enhancing the thermal stability of avidin. Introduction of disulfide bridges between subunit interfaces. J Biol Chem, 278, 2479-2483.
Paganelli, G., Chinol, M., Maggiolo, M., Sidoli, A., Corti, A., Baroni, S, and Siccardi, A. G. (1997) The three-step pretargeting approach reduces the human anti-mouse antibody response in patients submitted to radioimmunoscintigraphy and radioimmunotherapy. Eur J Nucl Med, 24, 350-351.
Paganelli, G., Grana, C., Chinol, M., Cremonesi, M., De Cicco, C., De Braud, F., Robertson, C., Zurrida, S., Casadio, C., Zoboli, S., Siccardi, A. G. and Veronesi, U. (1999) Antibody-guided three-step therapy for high grade glioma with yttrium-90 biotin. Eur J Nucl Med, 26, 348-357.
Paganelli, G., Magnani, P., Zito, F., VIIIa, E., Sudati, F., Lopalco, L., Rossetti, C., Malcovati, M., Chiolerio, F., Seccamani, E. and et al. (1991) Three-step monoclonal antibody tumor targeting in carcinoembryonic antigen-positive patients. Cancer Res, 51, 5960-5966.
Pazy, Y., Kulik, T., Bayer, E. A., Wilchek, M. and Livnah, O. (2002) Ligand exchange between proteins. Exchange of biotin and biotin derivatives between avidin and streptavidin. J Biol Chem, 277, 30892-30900.
Reznik, G. O., Vajda, S., Smith, C. L., Cantor, C. R. and Sano, T. (1996) Streptavidins with intersubunit crosslinks have enhanced stability. Nat Biotechnol, 14, 1007-1011.
Rosebrough, S. F. (1993) Pharmacokinetics and biodistribution of radiolabeled avidin, streptavidin and biotin. Nucl Med Biol, 20, 663-668.
Rosebrough, S. F. (1996) Two-step immunological approaches for imaging and therapy. Q J Nucl Med, 40, 234-251.
Rosebrough, S. F. and Hartley, D. F. (1996) Biochemical modification of streptavidin and avidin: in vitro and in vivo analysis. J Nucl Med, 37, 1380-1384.
Räty, J. K., Airenne, K. J., Marttila, A. T., Marjomäki, V., Hytönen, V. P., Lehtolainen, P., Laitinen, O. H., Mähönen, A. J., Kulomaa, M. S, and Ylä-Herttuala, S. (2004) Enhanced gene delivery by avidin-displaying baculovirus. Mol Ther, 9, 282-291.
Samols, D., Thornton, C. G., Murtif, V. L., Kumar, G. K., Haase, F. C. and Wood, H. G. (1988) Evolutionary conservation among biotin enzymes. J Biol Chem, 263, 6461-6464.
Schechter, B., Silberman, R., Arnon, R. and Wilchek, M. (1990) Tissue distribution of avidin and streptavidin injected to mice. Effect of avidin carbohydrate, streptavidin truncation and exogenous biotin. Eur J Biochem, 189, 327-331.
Thompson, J. D., Higgins, D. G. and Gibson, T. J. (1994) CLUSTAL W: improving the sensitivity of progressive multiple sequence alignment through sequence weighting, position-specific gap penalties and weight matrix choice. Nucleic Acids Res, 22, 4673-4680.
Weber, P. C., Ohlendorf, D. H., Wendoloski, J. J. and Salemme, F. R. (1989) Structural origins of high-affinity biotin binding to streptavidin. Science, 243, 85-88.
Wilchek, M. and Bayer, E. (1990) Introduction to avidin-biotin technology. Meth Enzymol, 184, 5-13.
Wilchek, M. and Bayer, E. A. (1999) Foreword and introduction to the book (strept)avidin-biotin system. Biomol Eng, 16, 1-4.
Wojda, U., Goldsmith, P. and Miller, J. L. (1999) Surface membrane biotinylation efficiently mediates the endocytosis of avidin bioconjugates into nucleated cells. Bioconjug Chem, 10, 1044-1050.
Wood, H. G. and Barden, R. E. (1977) Biotin enzymes. Annu Rev Biochem, 46, 385-413.
Yao, Z., Zhang, M., Sakahara, H., Nakamoto, Y., Higashi, T., Zhao, S., Sato, N., Arano, Y. and Konishi, J. (1999) The relationship of glycosylation and isoelectric point with tumor accumulation of avidin. J Nucl Med, 40, 479-483.
Yao, Z., Zhang, M., Sakahara, H., Saga, T., Arano, Y. and Konishi, J. (1998) Avidin targeting of intraperitoneal tumor xenografts. J Natl Cancer Inst, 90, 25-29.

1. An isolated protein which is structurally and functionally similar to with improved properties compared to native avidin or streptavidin and avidin-related proteins, AVRs, wherein the amino acid sequence of the protein has 40% or less, preferably about 30% or less homology, with avidin or streptavidin, and highly conserved fingerprint having the sequence:
WXN(E/Q/N/D)XGSX(M/L/F)X(I/V)X_{7,12}GX(F/Y) X_{17,36} (F/W)XVX(F/W)X_{3,10}(S/A)X(T/S)X(W/F)XGX_{5,14}(M/I/F/L)XXX(W/Y)X_{16,21}(D/N)XF, where in X denotes any amino acid residue; alternatives for a certain position are shown in parentheses, and the subscripted numbers indicate the lower and upper limit, respectively, for the length of the X-stretch in question.

2. The protein of claim 1 wherein the said protein is derived from Bradyrhizobium japonicum.

3. The protein of claim 2 having the amino acid sequence of SEQ ID NO:1 or SEQ ID NO:2.

4. The protein of claim 1 wherein the said improved properties comprise better affinity towards biotin conjugate, useful immunological properties, faster biotin dissociation rate or beneficial protein/protein-, protein/DNA or protein/ligand interaction or lack of it, compared to avidin and streptavidin.

5. A DNA sequence encoding the protein according to claim 1.

6. The DNA sequence of claim 5 having the sequence of SEQ ID NO:3 or 4, or a DNA sequence which hybridizes under stringent conditions with a DNA consisting of the nucleotide sequence of SEQ ID NO:3 or 4.

7. A recombinant vector comprising the polynucleotide sequence of claim 6.

8. A host cell comprising the recombinant vector of claim 7.

9. A recombinant protein produced by the host cell of claim 8.

10. A method for searching avidin-like proteins from databases or sequence libraries comprising use of a search string having the sequence:
WXN(E/Q/N/D)XGSX(M/L/F)X(I/V)X_{7,12}GX(F/Y)X_{17,36} (F/W)XVX(F/W)X_{3,10}(S/A)X(T/S)X(W/F)XGX_{5,14}(M/I/F/L)XXX(W/Y)X_{16,21}(D/N)XF, where in X denotes any amino acid residue; alternatives for a certain position are shown in parentheses, and the subscripted numbers indicate the lower and upper limit, respectively, for the length of the X-stretch in question.

11. A method to produce a medicament for targeted drug delivery, wherein the protein of claim 1 is used as an active ingredient.
